(19) United States
(12) Patent Application Publication
(10) Pub. No.: US 2002/0193846 A1
(43) Pub. Date: Dec. 19, 2002

(54) Instrumentation and software for remote monitoring and programming of implantable medical devices (IMDs)

(76) Inventors: Nancy Perry Pool, Minnetonka, MN (US); Craig M. Housworth, Woodbury, MN (US); Bozidar Ferek-Petric, Zagreb (HR)

Correspondence Address: MEDTRONIC, INC., 710 MEDTRONIC PARKWAY NE, MS-LC340, MINNEAPOLIS, MN, 55432-5604, US

(21) Appl. No.: 09/745,112

(22) Filed: Dec. 20, 2000

Related U.S. Application Data
(60) Provisional application No. 60/172,937, filed on Dec. 21, 1999.

(51) Int. Cl. A61N 1/08

(52) U.S. Cl. 607/60

(57) ABSTRACT

A modular system including hardware and software in combination or separately is used to adopt instruments for remote connectivity and programming of one or more medical devices in one or more patients. The modular system is implemented via an interface and is adaptable to a variety of medical devices, irrespective of origin of manufacture. The modular unit includes communication and other functional hardware in combination with Jini technology and Bluetooth implemented to effect wireless communication between various devices, patients and health providers.

This application claims priority to Provisional Patent Application No. 60/172,937 filed Dec. 24, 1999, and incorporates the specification and drawings in their entireties by reference herein.

### FIELD OF THE INVENTION

The invention generally relates to implantable medical devices (IMDs). Specifically, the invention relates to a bi-directional communications link between the IMDs and a remote expert data center implemented to chronically monitor and manage the IMDs associated with a patient in real time. More specifically, the invention relates to modular subsystems with add-on units interfaced with medical devices to enable remote monitoring and programming of the IMDs. These modules include instruments such as an RF head, telemetry interface units, ECG displays, touch screens and similar controls annexable to IMDs. Further a communication software applications program such as a Jini or equivalent is used for a remote method invocation, or RMI™. The software system is capable of using any network protocol that supports a compatible operating system. The invention enables programming of IMDs via the modular subsystems in cooperation with an instrument such as a programmer or an interface unit such as a PC, TV, VCR. The programmer or interface unit is preferably Web-enabled to communicate with various peripheral devices and computers locally and remotely.

### BACKGROUND OF THE INVENTION

Currently available implanted medical device remote monitoring and programming instruments for IMDs have several practical problems. Some of these problems include space-volume inefficiencies of instruments that take up valuable room in an already crowded medical clinic environment. Further, the design of these instruments appears to duplicate many of the electrical subsystems and operational functions provided by medical devices that are likely to already be available at clinics, exam rooms, operating rooms, emergency rooms, ambulances or medical helicopters.

Accordingly, many of the instruments duplicate the electrical subsystems and functions provided by other medical devices that are likely to already be available at these locations. Examples include ECG measurement from body surface electrodes, graphic displays, voice data connectivity, printer or printer port, touch screen and/or keyboard. The advent of widespread availability of low cost telecommunications technology, including internet based communications for medical care and therapy has improved problems of inefficiency resulting in an ever escalating cost in the health care system.

Specifically, new developments in telehealth and telemedicine require high levels of modularity among products and technologies. Telehealth is generally defined as a delivery of health care services from provider to patient via telecommunication links. Telemedicine, on the other hand, involves communications between providers such as consultation between primary care physicians and specialists, as well as on-line interaction between physicians and patients. This, and similar technologies, are intended to reduce overall cost of care and to improve access of patients to health care services. In the context of implanted medical devices, developing systems that allow patients to be monitored remotely in the home, and provide two-way interaction between the patient and the caregiver, require critical modular instrument technology as well as communication systems. This technology can potentially help reduce the number of home visits required and also provide more time in response to change in patient conditions. Specifically, remote patient management is of particular value for chronic disease patients. Telepathology also is an important emerging field and provides significant opportunities for providing advanced pathology services in the third-world countries from medical centers in the United States.

Various settings could be used for the delivery of telemedicine services, including the home, nursing home, rural clinics, schools, rural hospitals and the like. The systems are envisioned to provide direct contact with patients and primary care physicians as well as direct interaction between patients and specialists. This is particularly significant because of the shortage of specialists to be deployed in rural areas.

Anticipating this emerging trend, many existing instruments have or will incorporate many of the connectivity ideas disclosed in the present invention. For example, external defibrillators from PhysioControl, a division of Medtronic, already include a sophisticated remote connectivity built into them. Further, bedside monitoring systems, in particular systems that integrate a patient or patients with one or more medical devices, would require a modular programming and instrumentation system. More specifically, monitoring systems having the capability to program implantable medical devices such as those produced by Medtronic, without requiring the staff to go and retrieve a full featured programmer from the cardiology lab, would provide a significant cost, efficiency and operational advantage. Some of the generally connectivity-related instruments that are known in the art include a portable muscle stimulator, disclosed in U.S. Pat. No. 5,836,995 to McGraw. The stimulator has multiple independently driven channels connected to several corresponding electrodes for treating separate muscle groups of a patient. U.S. Pat. No. 5,289,824 to Homayoun et al, discloses a compact lightweight wrist-worn cardiac data and event monitor, the unit includes signal detection, data conversion, storage, display, telecommunication and external push-button control. Another instrument disclosed in the prior art relates to temporary pacemakers for control by a remote control programmer. U.S. Pat. No. 5,304,209 to Adams et al, discloses a pacemaker unit receiving control signals from a programmer and display unit displaying data relative to status for operation of a pacemaker unit with the fastener for temporary connection to a patient. The receiver receives control signals from the programmer. The display unit displays data relative to the status or operation of the pacemaker unit, and a fastener member fastens a temporary pacemaker to the body of the patient.

U.S. Pat. No. 4,142,533 to Brownlee et al discloses a telemetering and monitoring system for a cardiac pacer for controlling the testing of the functions of a pacemaker from a remotely located central facility. The disclosure includes provisions for directly and simultaneously transmitting from the pacer electrical signals indicative of multiple pacing functions. The indicative signals are picked up at the patient's location for local analysis and/or telephonically communicated to a remote central monitoring station. U.S. Pat. No. 4,203,448 to Keller discloses variable voltage multiplier for implanted cardiac pacemakers. The disclosure includes transistors operated by oscillator clocked counter to equal capacitor voltages. A memory system holds a program-controlled signal received from a remote source, and representing a desired multiplication factor of the supply voltage for pacer stimulation signals. U.S. Pat. No. 3,991,747 to Stanly discloses portable instruments for monitoring cardiac patients. The unit generally includes electrodes and control circuits for transmitting data to remote processing instruments. Signal processing system includes sensitive stable circuit elements providing low current and very high impedance provocation.

PCT publication WO/2000/27277 to Gopinathan et al, discloses a system for collecting diagnostic information and transmitting it to remote locations for providing emergency treatment. The invention includes two gloves that may be worn on a person's hands, the gloves including a number of diagnostic devices and a defibrillator device. The diagnostic devices are capable of sensing diagnostic signals from a person and the transmitting unit transmits information to, and receives information from, a remote location. The system may be used for obtaining medical diagnostic information and for gathering cardiac-related diagnostic information and transmitting the information from a remote location to a medical monitoring command center to provide both medical management information and emergency treatment to the patient at the remote location.

U.S. Pat. No. 6,052,624 to Mann, discloses a spinal cord stimulator system with electrodes capable of providing stimulation current for selectively stimulating specific areas based on directional signals and selected electrodes. Specifically, the invention provides a programming device that receives directional signals from a directional device to select a group of electrodes within an array for electrical stimulation so that the electrical stimulation current passing through selected electrodes enables stimulation areas to move with respect to the received directional signals. A pulse generator is provided with a programmable memory and receives a remotely generated programming signals for altering programmable memory for selectively applying electrical stimulation to two electrodes within the electrode array implanted within a patient.

U.S. Pat. No. 5,919,141 to Caldwell et al discloses a portable device for remote monitoring. Specifically the invention relates to vital sign monitoring of ambulatory patients in hospitals. Simultaneous monitoring of multi-channel ECG data, heart rate, pulse, oximetry, temperature, respiration and blood pressure is provided by a processor in a self-contained unit.

PCT Publication WO98/42407 to Nelson, C. G. et al, discloses an implantable device. The system includes a programmer at a patient station and an expert location with central computers. The implanted medical device is monitored and igested in the telepresence of remote experts having screen displays that mirror the displays at the patient locations. PCT Publication WO 98/42407 to Nelson C. G. et al discloses an implantable medical device remote expert communications system for co-ordinated implant and follow-up. The implantable medical device, monitoring and adjustment are enhanced by the telepresence of a remote expert having a screen display that mirrors the display at the patient location. EP Publication 856333 to Bottazzi et al, discloses a transtelephone system for monitoring and programming implantable cardiac pacemakers and defibrillators. The system includes at least one remote station connected to programming head of a cardiac pacemaker capable of receiving operating parameters of implanted devices at local station connected by telephone lines.

PCT publication WO96/11722 issued to Markowitz et al discloses a telemetry system for an implanted medical device. Specifically, the system includes a remote monitoring station, a repeater worn externally by a patient, and a quasi-passive transponder attached to a device implanted in the patient. The remote monitoring station communicates to the repeater to initiate an integration routine between the repeater and the transponder for extraction of patient information from the implanted device.

U.S. Pat. No. 5,487,755 to Mann et al, discloses a cardiac pacing remote operating system utilizing an external programming device which retrieves data from the implanted pacemaker. Specifically, the system involves establishing a telemetric link between a telemetry device of an external device and the telemetry circuit of a pacemaker. The information is downloaded into a memory on an external device, and an event record from the memory buffer of the pacemaker via the telemetric link with a telemetry circuit of the pacemaker.

U.S. Pat. No. 5,467,773 to Bergelson et al, discloses a pacemaker operation monitoring system. The instrument includes a local telephone setup to establish a two-way telephone connection. A local dual tone multi-frequency decoder responsive to dual tone multi-frequency signals received over the telephone line, generates respective local command signals. A patient monitoring portion is coupled to the telephone set. The monitor includes an amplifier, coupled to ECG leads. An ECG filter and a pulse filter pass ECG signals while surpressing a pulse signal. The system is used for remotely monitoring patients from a central station via a telephone network.

A further limitation of the prior art relates to the management of multiple medical devices in a single patient. Advances in modem patient therapy and treatment have made it possible to implant a number of devices in a patient. For example, an IMD, such as a defibrillator, a neural implant, a drug pump, a separate physiologic monitor, and various other IMDs may be implanted in a single patient. To successfully manage the operations and assess the performance of each device, in a patient with multiple implants, requires continuous updates and monitoring of the devices. Further, it may be preferred to have an operable communication between the various implants to provide a coordinated clinical therapy to the patient. Thus, there is a need to monitor. The IMDs, inlcuding the programmer on a regular, if not a continuous, basis to ensure optimal patient care. In the absence of other alternatives, this imposes a great burden on the patient. If a hospital or clinic is the only center where the necessary upgrade follow-up evaluation and digestment of the IMDs could be made. Further, if feasible, the situation would require the establishment of multiple service areas or clinic centers to support the burgeoning number of multi-implant patients world-wide. Accordingly, it is vital to have an instrument such as a programmer unit that is modular and would be able to connect the remote expert data center, all of the systems being alternate equivalents to provide access to an expert system and import the expertise to a local environment where the patient is located. Thus, there is a need for a modular unit that is both physically and electrically compatible with a variety of implantable medical devices to remotely monitor and program one or more implantable medical devices in one or more patients. Specifically, there is a need to reduce the total physical space needed by instruments in a medical setting where space is at a premium. Further, most programmers in remote connection systems duplicate many of the electrical subsystems and medical functions provided by other devices that are likely to already be available at patient stations and clinical centers. Furthermore, because of costs associated with programmers, it is expensive to equip various stations as well as clinical centers with programmers. Accordingly, there is a need to provide a modular system that is universally applicable and integrable with to various instruments and implantable medical devices while remaining functionally efficient and structurally simple, to promote remote communication and data exchange between medical devices and peripheral instruments.

### SUMMARY OF THE INVENTION

Generally, the invention discloses a system of a modularized package of software and hardware either in combination or separately implemented with at least one implantable medical device for remote monitoring and programming. The system is adaptable to existing medical equipment to reduce the total physical space needed, utilize common functional sub-systems and provide increased patient safety during remote programming.

Yet another aspect of the program includes combinations of subsystems implemented with an IMD, a remote monitor programmer, an external defibrillator, ECG monitor, a blood pressure monitoring instrument, a blood oxygenator instrument and any type of bedside operating room, emergency room or clinical physiological monitoring equipment which may include more than one of the instruments listed above.

Yet another aspect of the invention relates to the design of modules that would interface or plug into existing multifunction physiological monitoring stations used in hospitals, clinics or ambulances, thereby adding implantable medical device remote monitoring functionality to these stations without duplicating functions already provided by these stations.

An additional aspect of the invention includes the use of a highly diverse software system to transport information from the modules remotely to an expert station such as a clinical care provider using a dedicated software, for example, Java language and the Java Virtual Machine that would allow an applet to run on any platform. Specifically, the implementation might preferably use Jini as a way to make applets move transparently across networks regardless of the type of connection deployed. This software would be highly adaptable to the modular concepts disclosed in the present invention. For example, a code header that resides on top of Java applications would enable the network to move the application code just as it would move data. To the extent that an instrument is coupled to a network port, other instruments can communicate moving Jini-enabled applets across a network. The software system development contemplated by the present invention expands upon ongoing work on Java object repositories called Java Spaces and unites several other key Java technologies to enable networks which may encompass the entire Internet to become a giant virtual machine with a multiplicity of instruments and devices working together.

First stage implementation according to the present invention would be to have the Jini code about 25 KB in size, built into any instrument or device that can be connected to a network. Such devices might include hard drives, cameras, processors, displays or printers. Implementing such a code, the devices can offer services, for example, storage, over the network to others needing such a service. The present invention provides Jini software built on top of Java remote method invocation, RMI™. Jini enables the spontaneous networking of clients and services on the network. Both Jini and RMI hold a kind of directory service. In the case of Jini, the directory is called the Lookup Service. Jini provides a discovery protocol that enables clients to locate nearby lookup services without prior knowledge of their location. The Jini service object can use any network protocol to communicate back to any server, hardware or whatever, maybe across the network. Ultimately, a Jini service object could fully implement the service locally so that it need not do any communication across the network.

Accordingly, the present invention provides various modular systems that are adaptable to remote monitoring of one or more implanted medical devices in one or more patients, using software systems that are used at the patient station and a programmer station or central station. Accordingly, this invention provides interalia a modular system that is universally adaptable to provide remote communications between a patient station and a health care provider. More specifically, the invention enables simplicity and modularity in instrumentation and implanted medical device communication systems. Further, using instruments leveraged by both the modular hardware and software systems disclosed in the invention, a bi-directional wireless communication between patients (located at home or other centers) and their caregivers is enabled to monitor patients on a chronic full-time basis.

### BRIEF DESCRIPTION OF THE DRAWINGS

FIG. 1A is an illustration of a body implantable device system in accordance with the present invention, including a hermetically sealed device implanted in a patient and an external programming unit.

FIG. 1B is an illustration of a multi-implantable medical device system in accordance to the present invention, including various implanted medical devices in a patient having internal communication therein and also being communicable via instrumentation.

FIG. 2 is a perspective view of an external programming unit of FIG. 1A and FIG. 1B.

FIG. 3 is a block diagram of a typical implanted device of FIG. 1A or 1B.

FIGS. 4A, 4B and 4C depict various modular interface systems that are implemented in existing medical instruments in accordance with the present invention.

FIG. 5 is a block diagram showing implementations of Jini technology.

FIG. 6 is a block diagram illustrating the application Jini technology together with home audio-visual systems.

FIG. 7 is a block diagram illustrating the application of Jini technology to a simplified modular bedside programmer in combination with an audio-visual system such as a VCR.

FIG. 8 is a block diagram illustrating the application of Jini technology to an instrument such as ECG recorder in combination with an audio-video system.

FIG. 9 is a block diagram illustrating the application of Jini technology to a programmer and a data reporting and printing system.

FIG. 10 is a block diagram illustrating the application of Jini technology in cooperation with an implanted medical device and a home PC.

### DETAILED DESCRIPTION OF THE DRAWINGS

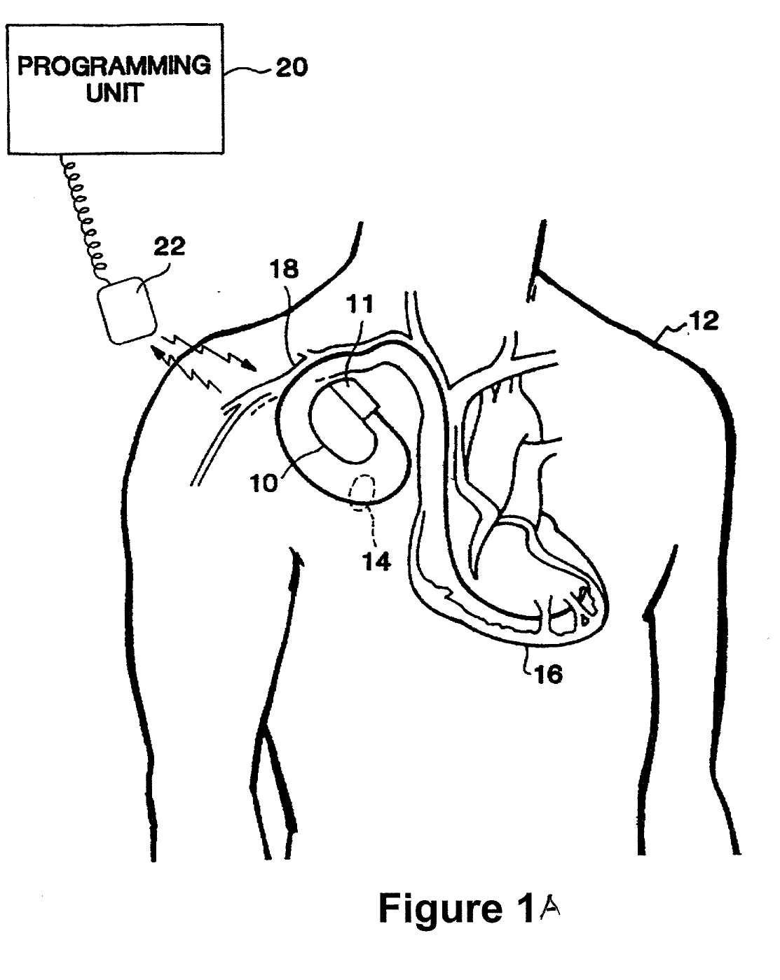

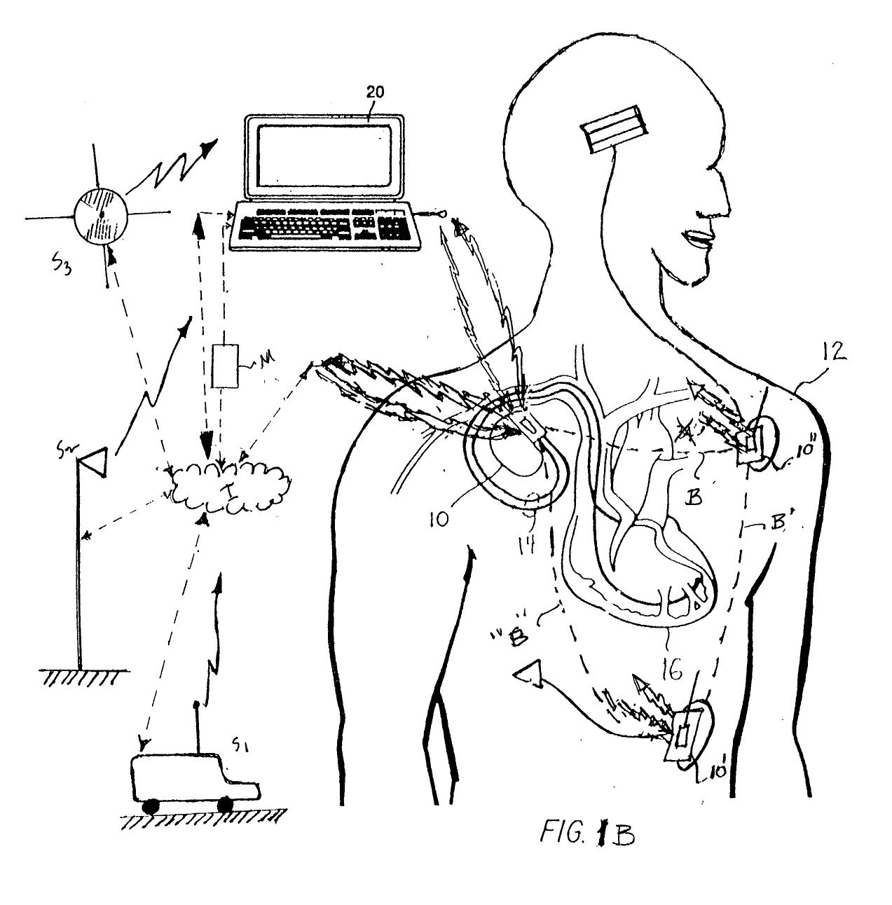

FIG. 1 is an illustration of an implantable medical device system adapted for use in accordance with the present invention. The medical device system includes an IMD 10 implanted in a patient 12. A ventricular pacemaker lead 14 is electrically coupled to pacemaker 10 in a conventional manner and extends into the patients heart 16 vein 18. Near the distal end of lead 14 are one or more conductive electrodes for receiving electrical cardiac signals and/or for delivering electrical pacing stimuli to heart 16. Also depicted in FIG. 1 is an external programming unit 20 for noninvasive communication with implanted device 10 via uplink and downlink communication channels. Associated with programming unit 20 is a programming head 22 for facilitating two-way communication between implanted device 10 and programmer 20.

FIG. 1B is an alternate embodiment of FIG. 1A wherein several implantable medical devices, for example 10, 10′ and 10″ are implanted in patient 12. The devices may have internal communication within (B, B′ and B″) patient 12 and individual telemetric communication with programmer 20. In the alternate, the devices may have a common communication channel with programmer 20. Several other communication systems are disclosed wherein telecommunications could be implemented to provide a wireless communication between various stationary and mobile stations. For example, S1, S2, S3 represent a mobile station, a stationary station and a satellite system respectively. The system may also enable direct communication between programmer 20 and the Internet via modem M.

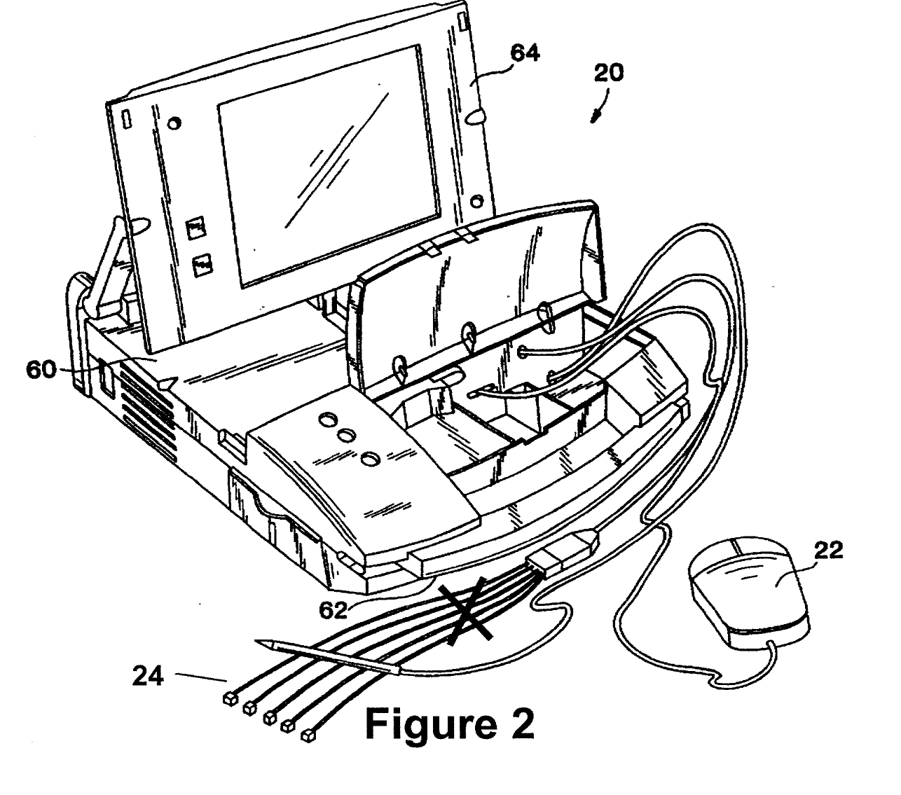

FIG. 2 is a perspective view of programmer 20 in accordance with the presently disclosed invention. Internally, programmer 20 includes a processing unit not shown in the figure that, in accordance to the presently disclosed invention, is a personal computer type motherboard, for example, a computer mother board including a microprocessor such as an Intel Pentium III and related circuitry such as digital memory. The details of design and operation of the programmer's computer system will not be set forth in detail in the present disclosure as it is believed that such details are well known to those of ordinary skill in the art. Still referring to FIG. 2, programmer 20 includes an outer housing 60 and a carrying handle 62 so programmer 20 can be carried like a briefcase. An articulating display screen 64 is disposed on the upper surface of housing 60. As would be appreciated by those of ordinary skill in the art, display screen 64 is operatively coupled with computer circuitry disposed within housing 60 and is adapted to provide a visual display of graphics and/or data under the control of the antenna computer. As would be appreciated by those of ordinary skill in the art, it is often desireable to provide a means for determining the status of the patient's conduction system. Normally, programmer 20 is equipped with external ECG leads 24. In accordance with the present invention, programmer 20 is equipped with an internal printer (not shown) so that a hard copy of the patient's ECG or of graphic displays on the programmer's display screen 64 can be generated.

Several types of printers, such as the AR100 printer, available from General Scanning Company are known and commercially available to work with programmer 20. Programmer 20 described herein with reference to FIG. 2 is disclosed in more detail in U.S. Pat. No. 5,345,362, issued to Thomas J. Winkler, entitled “PORTABLE COMPUTER APPARATUS WITH ARTICULATING DISPLAY PANEL”, which patent is hereby incorporated herein by reference in its entirety. The Medtronic Model 9790 Programmer is an implantable device programming unit with which the present invention may be practiced.

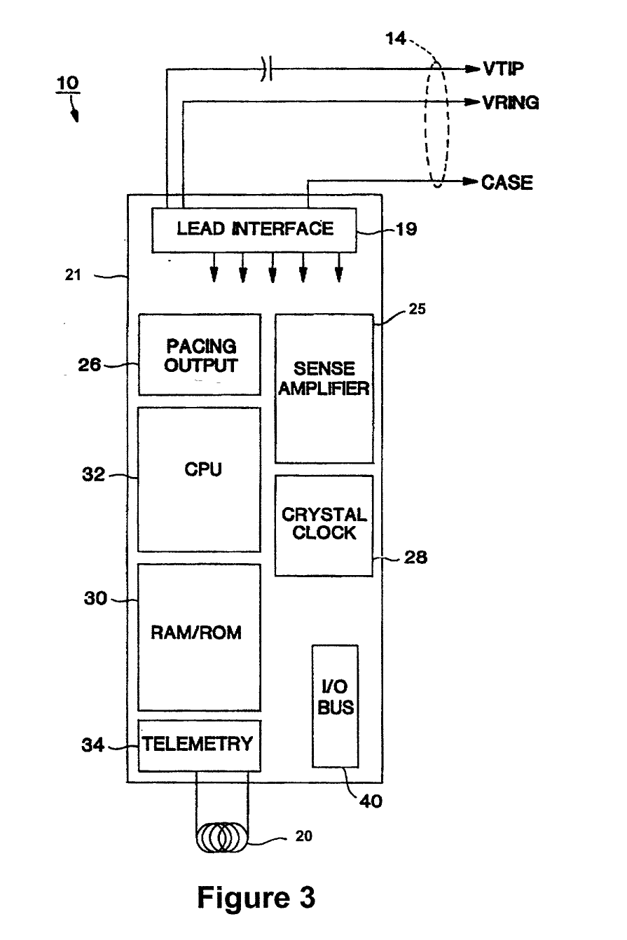

FIG. 3 is a block diagram of the electronic circuitry that makes up pulse generator 10 in accordance to the presently disclosed invention. As can be seen from FIG. 3, generator 10 comprises a primary simulation control circuit 21 for controlling the device's pacing and sensing functions. The circuitry associated with stimulation control circuit 21 may be of conventional design in accordance, for example, with what is disclosed in U.S. Pat. No. 5,052,388 issued to Sivula et al, entitled “METHOD AND APPARATUS FOR IMPLEMETNING ACTIVITY SENSING IN A PULSE GENERATOR”. To the extent that certain components of pulse generator 10 are conventional in their design and operation, such components will not be described herein in detail, as it is believed that design implementation of these components would be a matter of routine to those of ordinary skill in the art. For example, stimulation control circuit 21 of FIG. 3 includes stimulating pulse output circuit 26, a crystal clock 28, random access memory and read only memory (RAM/ROM) unit 30 and a central processing unit (CPU) 32, all of which are well known in the art. Pacemaker 10 also includes internal communication circuit 34 so that it is capable of communicating with internal programmer/control unit 20 as described in FIG. 2 in greater detail. Specifically circuit 34 relating to telemetry, the particular focus to the present invention because most of the wireless communication system and the schemes implemented by the present invention are interfaced with the implanted medical device via this internal communication circuit 34.

With continued reference to FIG. 3, pulse generator 10 is coupled to one ventricular lead 14 which, when implanted, extends transvenously between the implant site of post generator 10 and the patient heart 16 as previously noted with reference to FIGS. 1A and 1B. Physically, the connections between lead 14 and the various internal components of post generator 10 are facilitated by means of a conventional connector block assembly 11 shown in FIG. 1. Electrically, the coupling of the conductors of lead 14 and internal electrical components of pulse generator 10 may be facilitated by a lead interface circuit 19 which functions in a multiplexor like manner to selectively and dynamically establish necessary connections between various conductors and leads 14 including ventricular tip and ring electrode conductors and individual electrical components of post generator 10 as is familiar to those of ordinary skill in the art.

For the sake of clarity, the specific connections between lead 14 and the various components of post generator 10 are not shown in FIG. 3, although it will be clear to those of ordinary skill in the art. For example, that lead 14 will necessarily be coupled either directly or indirectly to sense amplifier circuitry 25 and the simulating pulse output circuit 26 in accordance with common practice such that cardiac electric signals may be conveyed to sensing circuitry 25 to enable the delivery of stimulating pulses to cardiac tissue via leads 14. Also not shown in FIG. 3 is protection circuitry commonly included in implanted devices to protect, for example, the sensing circuitry of the device from high voltage stimulating pulses. Stimulating control circuit 21 includes central processing unit 32 which may be an off-the-shelf microprocessor or microcontroller, but in the present invention could be a custom integrated circuit. Although specific connections between CPU 32 and other components of stimulation control circuit 21 are not shown in FIG. 3, it should be apparent to those skilled in the art that CPU 32 functions to control the timed operation of stimulating pulse output circuit 26 and sense amplifier circuit 25 under control of programming stored in RAM/ROM unit 30. It is believed that those of ordinary skill in the art will be familiar with such an operative structure and arrangement. With continued reference to FIG. 3, crystal off letter 28 provides mean timing cross signals to stimulation control circuit 21. Again, the lines over which such crossing signals are provided to the various timed components of pulse generator 10 are omitted from FIG. 3 for the sake of clarity. It is to be understood that the various components of post generator 10 depicted in FIG. 3 are powered by means of a batter that is contained within the hermetic enclosure of pacemaker 10 in accordance with common practice in the art. For the sake of clarity in the figures, the battery and the connections between it and the other components of post generator 10 are not shown. Stimulating post output circuit 26, which functions to generate cardiac stimuli under control of signals issued by CPU 32 may be, for example, of the type disclosed in U.S. Pat. No. 4,476,868 to Thompson, entitled “BODY STIMULATOR OUTPUT CIRCUIT”, which patent is hereby incorporated by reference in its entirety. Again, however, it is believed that those of ordinary skill in the art could select from many different types of prior art pacing output circuits that would be suitable for purposes of practicing the present invention.

Sense amplifier circuit 25 functions to receive electrical cardiac signal from ventricular lead 14 and to process such signals to derive event signals reflecting the occurrence of a specific cardiac electrical event. CPU 32 provides this event indicating signal for use in controlling the synchronous stimulating operation of post generator 10 in accordance with common practice in the art. In addition, this event indicating signals may be communicated by an uplink transmission to external programming unit 20 for visual display to a physician or clinician. Those of ordinary skill in the art will appreciate that pacemaker 10 may include numerous other components and systems. For example, activity sensors and associated circuitry. The presence or absence of such additional components in pacemaker 10, however, is not believed to be pertinent to the present invention which relates primarily to the implementation or remote communication, preferably via circuitry 25 in pacemaker 10 and associated communications in external units such as programmer 20.

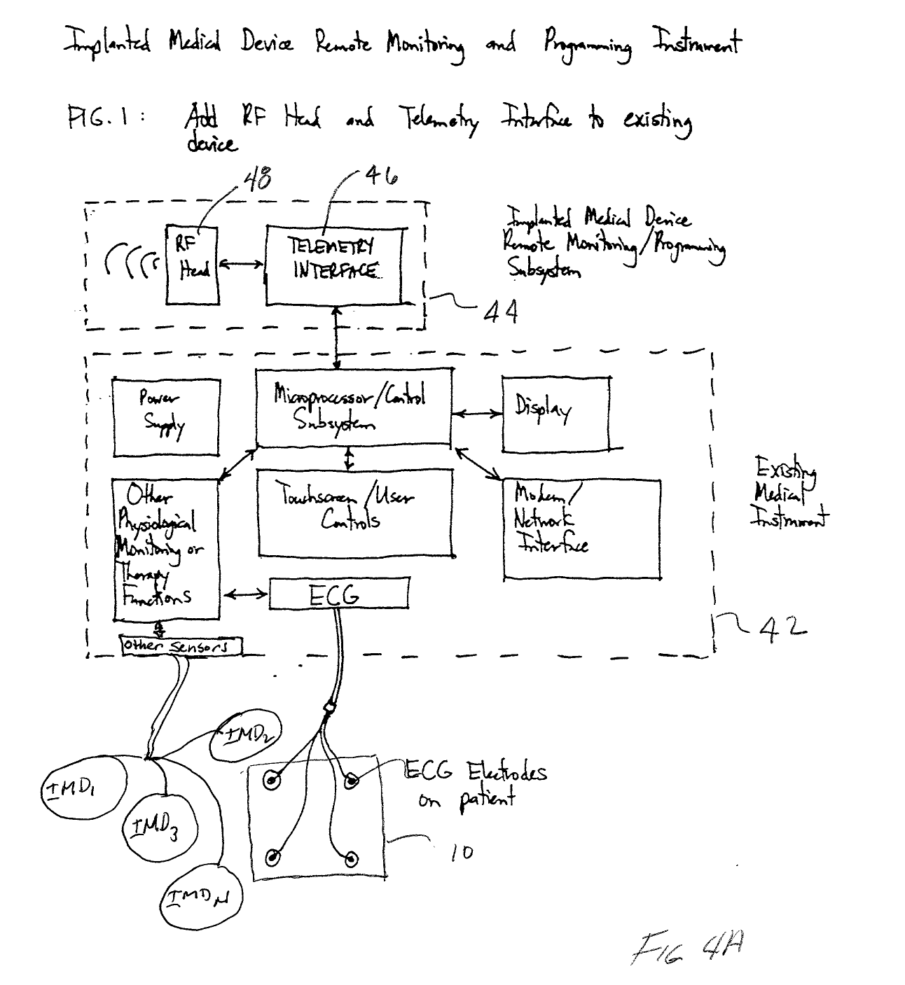

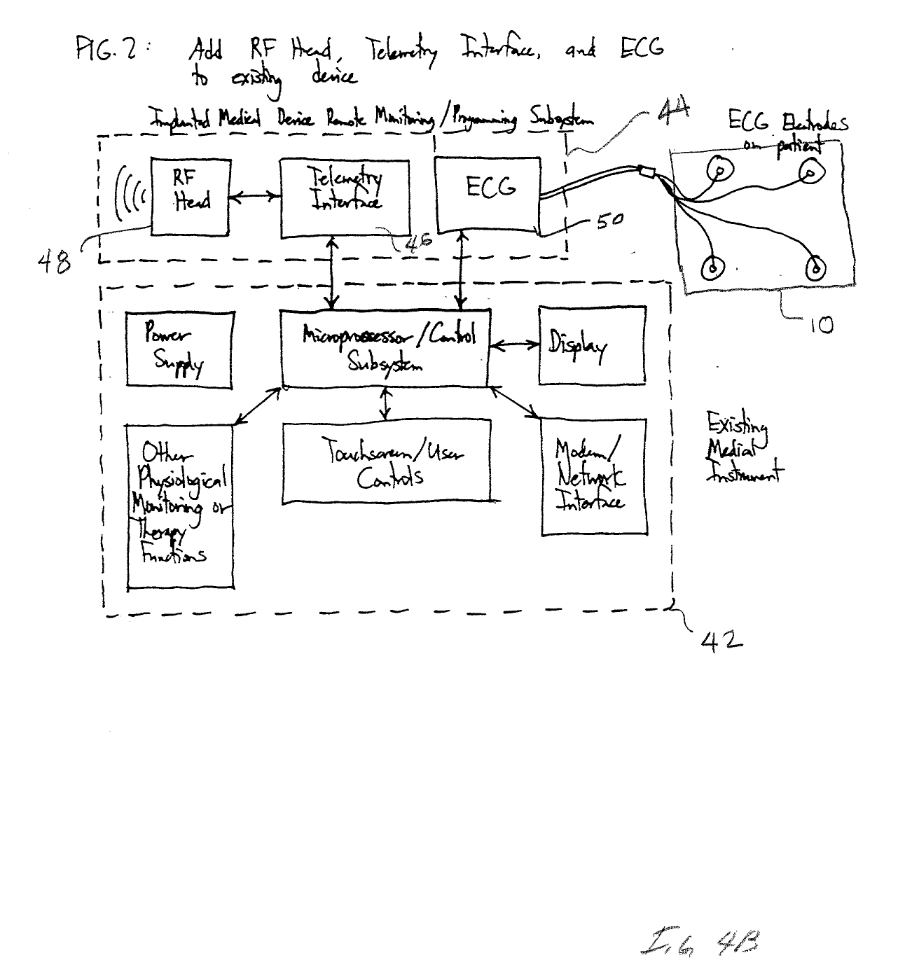

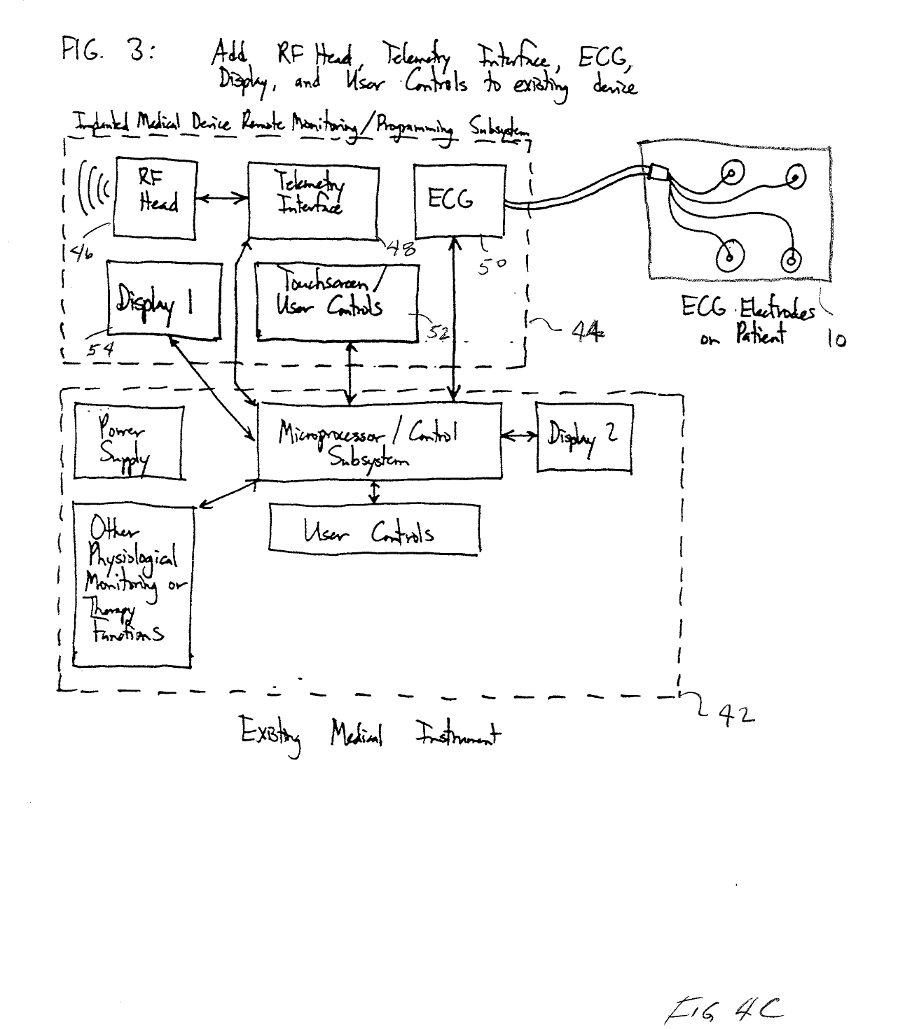

FIG. 4A represents an implanted medical device remote monitoring instrument. Specifically, existing medical instrument 42 is illustrated having a power supply, microprocessor/control system, touch screen/user control displays, modem/network interface and other physiological monitoring or therapy functions such as ECG. Those of ordinary skill in the art will appreciate that existing medical instrument 42 may include numerous other components and subsystems depending upon the implementation and operation of the medical instrument. If for example the instrument is a pacing device, it will have an ECG component with ECG electrodes providing connections to the patient via an implanted medical device. Similarly, other components may be implemented in multi-implant environments such as shown in FIG. 1B wherein multiple implantable medical devices provide connections to the patient. In the context of the present invention, a remote monitoring/programming subsystem 44 is connected to existing medical instrument 42 to provide telemetry interface 46 and RF head 48. Accordingly, implanted medical device remote monitoring/programming subsystem 44 enables wireless transfer of data from existing medical instrument 42 to a remote station as needed. As discussed hereinbelow, microprocessor/control subsystem of existing medical instrument 42 may be programmed to enable specific data transfer and receipt from remote monitoring/programming system 44.

FIG. 4B is a variation of FIG. 4A wherein remote subsystem 44 includes an ECG data management system which could be coupled directly to implanted medical device 10. In this arrangement, existing medical instrument 42 would exchange data with implanted medical device 10 via remote subsystem 44.

FIG. 4C is yet another variation of FIG. 4A in which remote monitoring programming/subsystem 44 includes ECG 50, touch screen user control 52 and display 54. Specifically ECG 50 is connected directly to implanted medical device 10 to enable data transfer to remote monitoring/programming subsystem 44. Similar to the disclosure in FIG. 4B, in this arrangement, medical instrument 42 would exchange data and communicate with implanted medical device 10 via remote monitoring/programming system 44.

Accordingly, as indicated and shown in exemplary FIGS. 4A, 4B and 4C, remote monitoring/programming subsystem 44 would be structured to accommodate various arrangements for either direct uplink of patient data from implanted medical devices such as implanted medical device 10 or to transfer medical data and information from existing medical instruments such as 42 as illustrated in FIG. 4A. In one aspect of the invention, remote subsystem 44 could be modularized to hook up to a number of instruments including implanted medical devices to enable highly flexible and tailorable compact modular system that is space and volume efficient to work with existing medical instruments 42. Further, the implanted medical device, could be connected, via subsystem 44 to a remote expert data center, a remote Web-based data center or remote data center, all being alternate equivalents as used herein, to provide remote communications and monitoring.

Further, it is important to have a local program operator/manager technician who could be trained remotely by exporting software based training regimen from a remote Web-based center with automated features to provide onsite training, specification generation, specification notification and other enabling software. More specifically, it is most desireable to provide globally distributed technicians or programmers a software based system that could be used for upgrading and transferring data including training consistent with the standards set by the manufacturer of the implanted medical device and the programmer, as well as in compliance with specification regulation of the country in which the technician or operator is located.

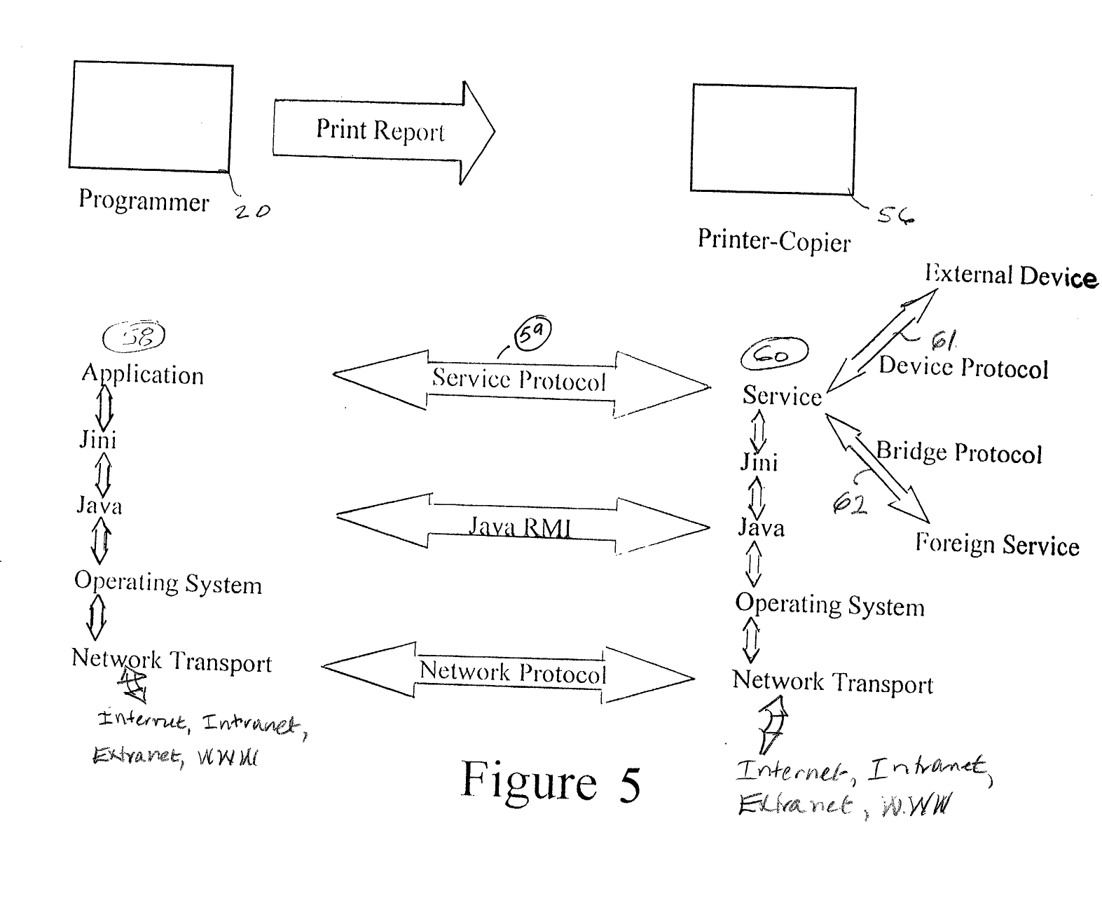

FIG. 5 illustrates one of the numerous possible ways in which Jini technology is implemented in an instrument such as programmer 20. The Left Column 58 represents the software application program. The Center Column 59 represents the device that opens the service. The Right Column 60 shows the service and whether other devices need to be accessed via device protocol 61 and bridge protocol 62. The arrow indicates the different levels of communication that are required. Consistent with the present invention, a modular subsystem could be added to programmer 20. Specifically, a software program written in Java language with a Jini header is implemented. The operating system will use a network transport. Similarly, printer copier 56 will be defined in Java code with a Jini on top of it. A modular subsystem, such as subsystem 44, would enable programmer 20 to locate the printer service via remote interface (for example, telemetry interface 46) and Jini technology, which employs Java Remote Method Invocation (RMI™). The RMI™ utilizes the network protocol that is supported by the operating system. Subsystem 44 added to programmer 20 may, for example, include an infrared network interface, wireless radio-frequency network, or a plug-in modem or equivalent to access the network. Thus, instead of building a printer with programmer 20, a subsystem 44 hardware and Jini technology would enable the use of an external device such as printer-copier 56 remotely or locally.

Still referring to FIG. 5, programmer 20 locates printer 56 by using Jini technology. Thereafter, programmer 20 downloads and runs the Java code supplied by the printing service. The code uses the underlying network transport to implement the printing service protocol needed to transmit the follow-up report to printer 56.

After programmer 20 locates printer service 60 to print a report at printer copier 56, the program runs the Java codes supplied by printing service 60. This code uses the underlying network transport and, preferably RMI™ technology, implements the printing service protocol needed to transport or transmit the follow-up report to the printer. The service protocol that an application and the service uses to communicate to each other can either be an existing protocol or a new one defined by the manufacturer providing the service. Some of the emerging network technologies are based on new service protocols that are more intelligent and flexible than current ones. These are all compatible with Jini technology. A few of the emerging network technologies define their own protocols to locate and communicate with devices such as device protocol 61. For this, the Jini service needs to act as a bridge. Such a bridge protocol 62 involves, generally, translating the application's request into the protocol used by the other network and forwarding it to the device. This requires that service 60 be operated on a system/device such as printer 56 and programmer 20, that is also connected to the other networks under network transport.

FIG. 5 further illustrates the application of Jini technology together with home audio/video operability as indicated by bedside programmer 62 and television set 64. It is assumed that TV set 64 complies with specifications for home network of consumer electronic devices such as CD players, VCRs, digital cameras and set top boxes. In this system, the network configuration is automatically updated as devices are plugged in or removed. Application 58 is designed to coordinate the control of several devices and to simplify the use of the devices by the user. Home audio, video, interoperability, HAVi network is an example of where a bridge protocol 62 would be required to provide a gateway for shared services between HAVi devices and devices using Jini technology. Applications using Jini software can be used to access to HAVi devices such as TV set 64. TV set 64 could connect to remote Jini services 60 via application 58 based on video on demand operations.

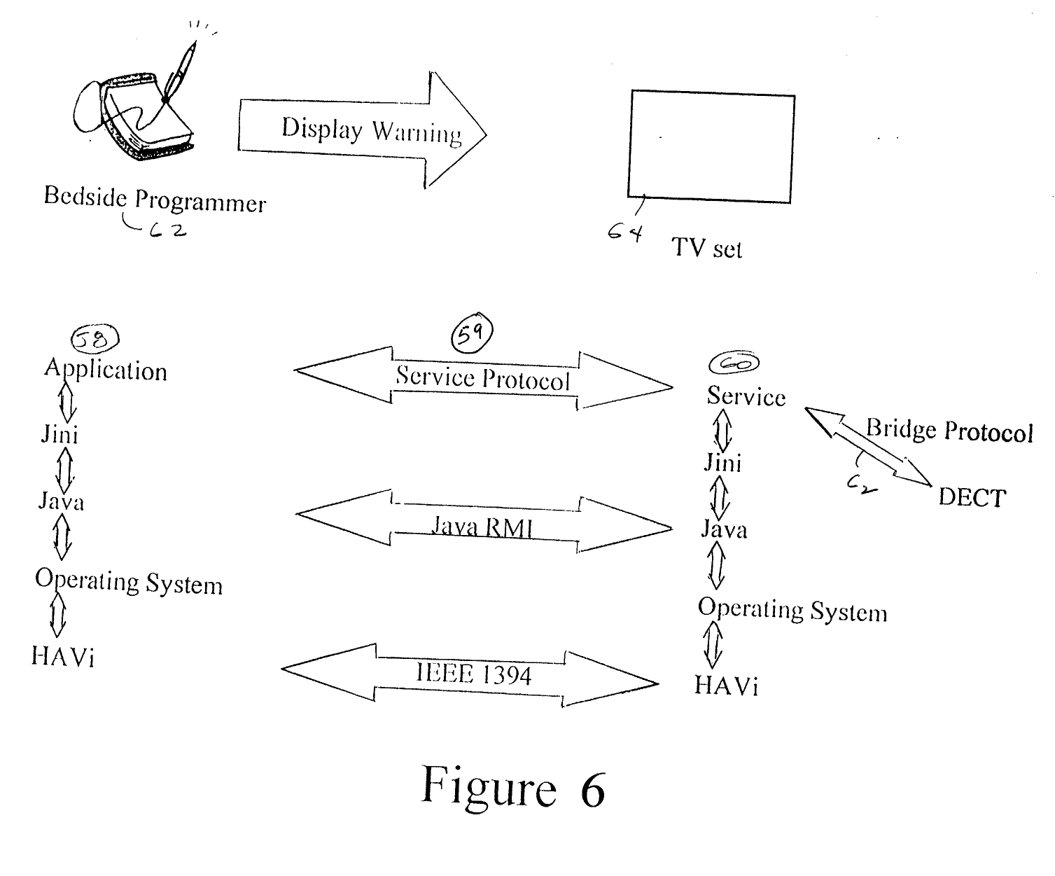

FIG. 6 illustrates, a bedside programmer 62 having telemetry link with an implanted medical device (not shown). Bedside programmer 62 includes an IEE1394 interface. The implanted medical device, for example, may issue a patient alert or may turn on the FASC indicator. Based on the understanding that patients are likely to watch TV than to listen to their implanted medical device alert signal, in the arrangement shown in FIG. 6, TV set 64 may display a patient alert on the TV screen. The alert may be a warning or a signal to the patient to review a status of a scheduled follow-up session. In the alternate, the entire system may be connected to the home DECT terminal. In this arrangement, the message may be sent to the patient's counseling physician who may respond with a message on TV set 64 instructing the patient about the alert conditions.

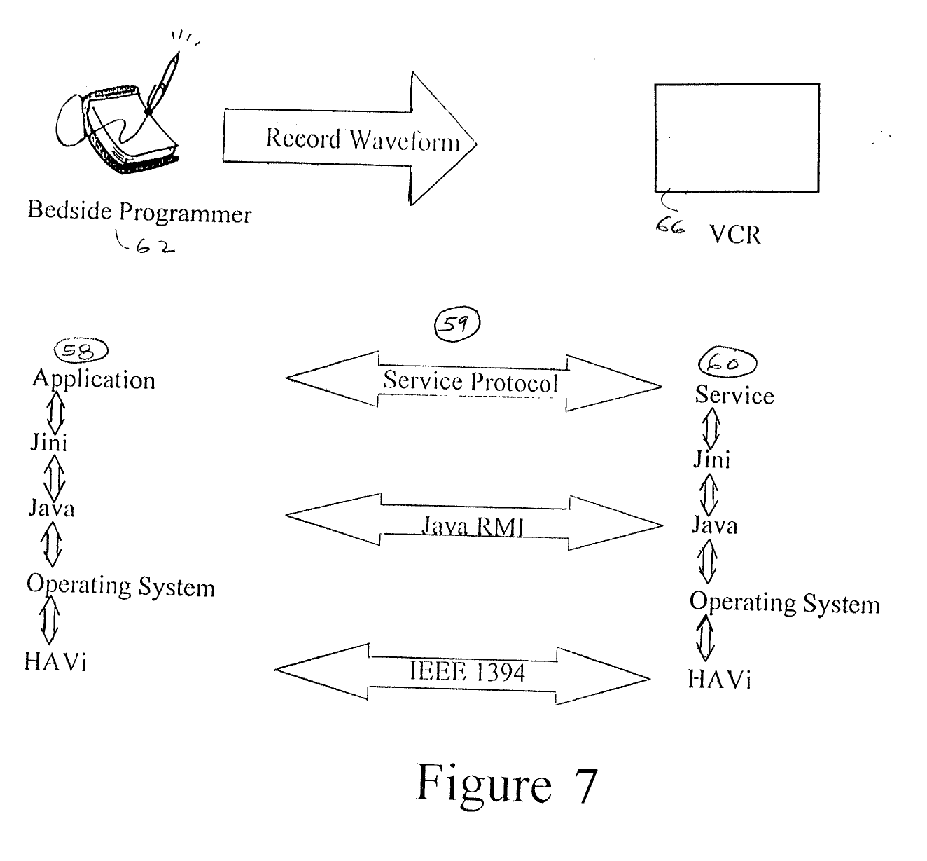

Referring to FIG. 7, another aspect of Jini and HAVi bedside programmer 62 is disclosed. This arrangement provides high level storage capability. Specifically, VCR 66 is used for recording various signals utilizing FM and high fidelity audio signals. In the same manner, VCR 66 may be used to record ECG signals or other physiological data from an implanted medical device. Specifically, an implanted medical device such as IMD 10 that may detect arrhythmia and restore the preventative segments of EGM in its memory may be used to transmit to bedside programmer 62 recording of the signals on VCR 66. As indicated in FIG. 1B, various implantable medical devices in a patient may communicate using Bluetooth adapted for use in patients. Similarly, Bluetooth could be adapted for use in bedside programmer 62 in combination with DDAs, laptops, mobile phones and other portable devices. Those skilled in the art would know that when Bluetooth devices come close together, they automatically detect each other and establish a network connection. This unique feature could be implemented in a network transport protocol for use to allow instruments using Jini technology to communicate without being physically connected to each other. Other technologies like PIANO, which can be built on top of Bluetooth, could be used to specify the type of information that the instruments may exchange and how they communicate within the wireless network. These and other operating systems like EPOC32 for cell phones provides the necessary features to support Jini technology.

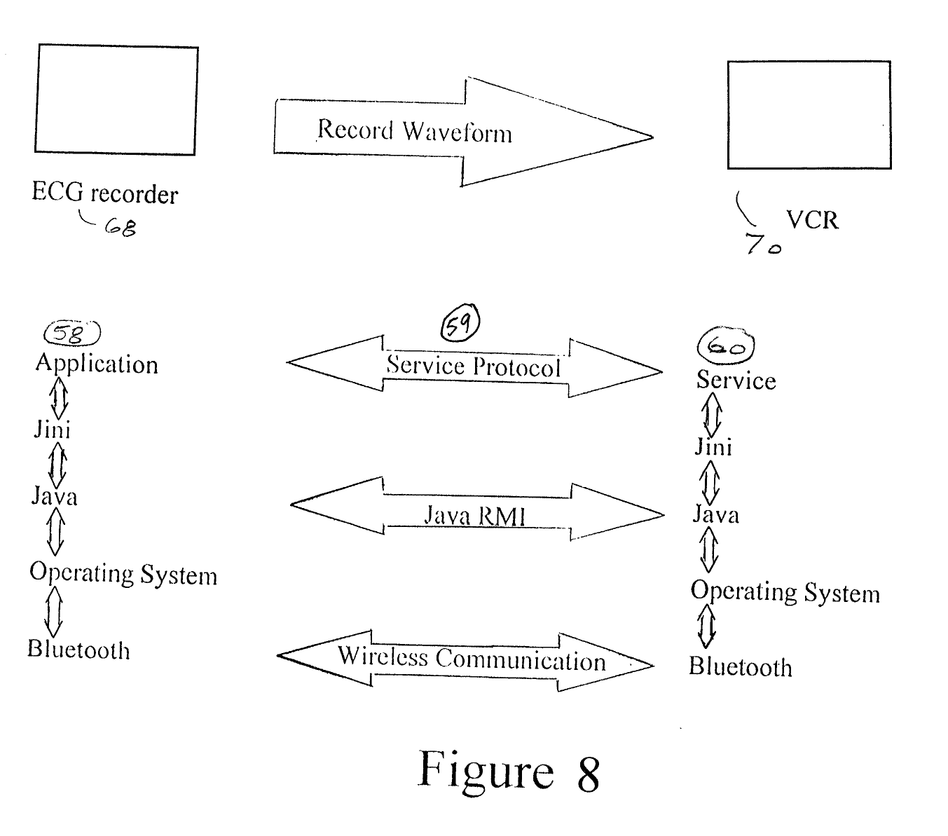

FIG. 8 illustrates a wireless ECG recorder 68 utilizing VCR 70 as a recording medium. The lookup service is a waveform recording and is offered by VCR 70. Jetsend technology, for example, as provided by Hewlett Packard, is a service protocol that allows peripheral equipment like printers, digital cameras and PCs to intelligently negotiate information exchange without user intervention. The Jetsend protocol allows the devices to identify a common data format and exchange data on that basis. Once Jini technology has been used to connect the recorder 68 and VCR 70, the Jetsend protocol can be used to transfer information between them. Accordingly, in FIG. 8, ECG recorder 68, that may be a modular unit consistent with the present invention, could be integrated with VCR 70 to record all ECG data using Jini technology.

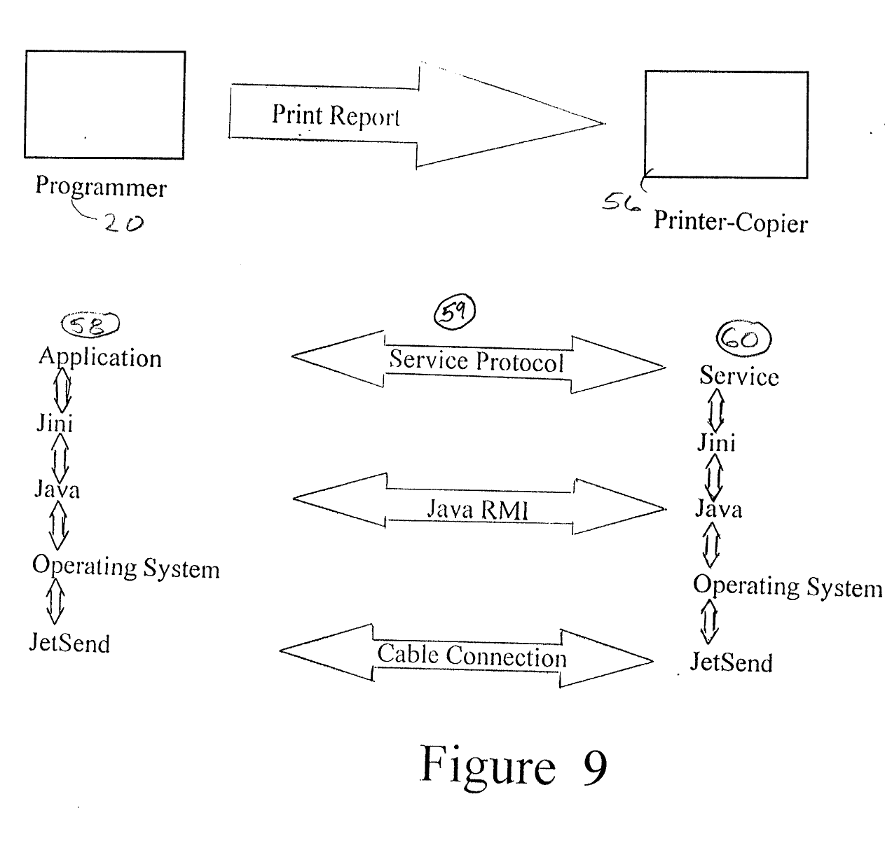

FIG. 9 is an alternate embodiment of the disclosure of FIG. 8 where programmer 20 is serviced by printer copier 56 to print report consistent with Jini technology under application 58 and service mode 60. As indicated, Jetsend protocol allows the devices to use a common service protocol with Java RMI using Jetsend as an operating system via cable connection or similar data transfer systems.

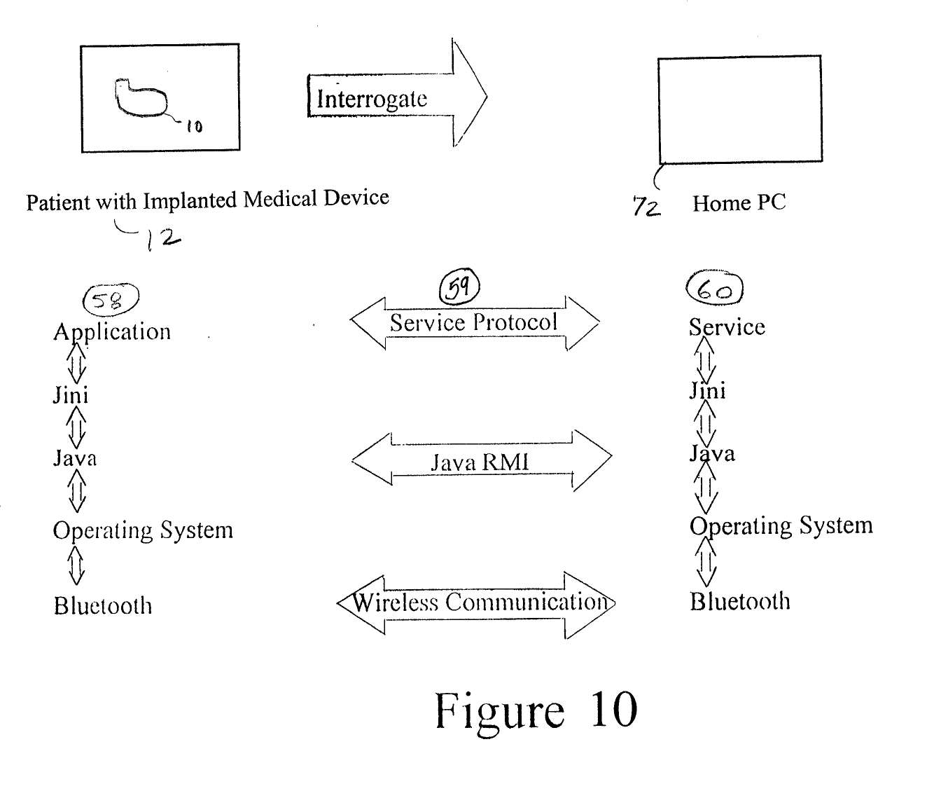

FIG. 10 illustrates an application in which an implantable medical device 10 in patient 12 initiates an interrogation process to obtain a look up service via home PC or computer 72. Utilizing wireless Bluetooth technology, for example, home PC 72 can interrogate the basic data displayed on a quick look screen to warn patient 12 about arrhythmias or other physiological events.

Accordingly, the present invention provides modular solutions for existing medical instruments. Specifically, remote monitoring or programming subsystems, as indicated in FIGS. 4A through 4C, are adapted to enhance/expand the functionality of instruments. More specifically, Jini technology may be implemented to extend information transfer and exchange remotely between patients at home and their service providers. Under the structure and software scheme of the present invention, instruments such as programmers may be enabled to use remote printers and copiers. Highly simplified bedside modular programmer may be integrated with TV sets to display warning signals, or to display and record waveforms on a VCR. Additionally, ECG data from a medical device could be directly displayed and recorded on a VCR using Bluetooth and Jini technology.

It is to be understood that the modular method, structures and software of the present invention provide for modification and modularity of existing instruments regardless of the source of manufacturer. The scheme advanced in the present invention enables universal adaptability of instruments to use existing devices to promote remote patient monitoring and communication systems.

It is to be understood that the above description is intended to be illustrative and not restrictive, meaning other embodiments would be apparent to those of skill in the art upon reading and understanding the above description. The scope of the invention should therefore be determined with reference to the appended claims along with the full scope of equivalence to which such claims are entitled.

What is claimed is:

1. A modular system implemented to adapt instruments for remote connectivity in conjunction with one or more implantable medical devices, the modular systems comprising:
a remote monitoring/programming subsystem;
an interface unit, and
at least one medical instrument; said interface unit providing connections between said subsystem and said instrument to add the remote connectivity to the instrument.

2. The system of claim 1 wherein said subsystem includes modular elements.

3. The system of claim 2 wherein said modular elements are aggregates of one or more functional units integrated within said subsystem.

4. The system of claim 3 wherein said aggregates of one or more functional units include independent add-on features adaptable for universal integration with said instruments.

5. A modular system including a software system adapted to one or more implantable devices in one or more patients to enable remote monitoring, communication and programming of the one or more patients, the system comprising:
a remote monitoring/programming subsystem integrated with the one or more medical devices; and
a software system implemented to provide network connectivity to said subsystem.

6. The system of claim 5 wherein said software system includes Jini Technology.

7. The system of claim 6 wherein said Jini technology implements a Java language.

8. The system of claim 6 wherein said Jini technology implements a Java RMI™.

9. The system of claim 8 wherein said Jini technology wherein Jetsend is used as our operating system to use a common or common service protocol with Java RMI™.
